(19) United States
(12) Patent Application Publication
(10) Pub. No.: US 2006/0024247 A1
(43) Pub. Date: Feb. 2, 2006

(54) Oral care formulations comprising highly bioavailable Coenzyme Q-10 cyclodextrin complex

(75) Inventors: Doddabele L. Madhavi, Worcester, MA (US); Daniel I. Kagan, Belmont, MA (US)

Correspondence Address: Jerry K. Mueller, Jr.;Mueller and Smith, LPA, 7700 Rivers Edge Drive, Columbus, OH, 43235, US

(73) Assignee: BioActives, LLC

(21) Appl. No.: 11/190,094

(22) Filed: Jul. 26, 2005

Related U.S. Application Data
(60) Provisional application No. 60/591,970, filed on Jul. 28, 2004.

(51) Int. Cl. A61K 9/68; A61K 8/66

(52) U.S. Cl. 424/49; 424/48

(57) ABSTRACT

The present invention incorporates of a highly bioavailable coenzyme Q-10/cyclodextrin inclusion complex into oral care products, such as, toothpaste, mouth wash, chewing gum, breath mint, mouth spray, gels, and lozenges. The inclusion complex also is suitable for devices, such as dental loops, for delivering coenzyme Q-10 to the periodontum by direct physical contact. The inclusion complex is water dispersible or water soluble, stable in the presence of components of the formulation, and is highly bioavailable to the cells in the oral cavity.

### CROSS-REFERENCE TO RELATED APPLICATIONS

This application claims priority from provisional application serial No. 60/591,970 filed Jul. 28, 2004; and is cross-referenced to commonly-owned application Ser. No. 10/748,096 filed Dec. 30, 2003, the disclosure of which is expressly incorporated herein by reference.

### STATEMENT REGARDING FEDERALLY SPONSORED RESEARCH

Not applicable.

### BACKGROUND OF THE INVENTION

This invention relates to incorporation of highly bioavailable Coenzyme Q-10/cyclodextrin complexes in to oral care formulations and devices.

Coenzyme Q-10 (hereinafter, “CoQ-10”) or ubiquinone is a water insoluble antioxidant involved in electron transport and oxidative phosphorylation. Several human studies have indicated that CoQ-10 is necessary for the maintenance of healthy gums and teeth. A deficiency of CoQ-10 has been observed in gingival tissues of individuals with gum disease (Littarru, G. P., Nakamura, R., Folkers, K., and Kuzell, W. C., “Deficiency of Coenzyme Q-10 in gingival tissue from patients with periodontal disease”, Proc. Nat Acad. Sci., 68: 2332-2335, 1971). Topical application of CoQ-10 has been reported to regenerate damaged gum tissue of patients with periodontal disease (Hanioka, T., Tanaka, M., Ojima, S., Shizukuishi, S., and Folkers, K., Molec. Aspects Med., 15 (Suppl): s241-s248, 1994). CoQ-10 apparently facilitates the healing process by increasing the production of energy in the infected tissue and improving the immune system. CoQ-10 also may provide such benefits based on its antioxidant properties.

CoQ-10 is a lipophilic molecule practically insoluble in water. The bioavailability of CoQ-10 is very poor and related to the rate dissolution in formulations. Several oral care compositions containing solubilized CoQ-10 have been disclosed (U.S. Pat. No. 6,200,550; U.S. Pat. No. 6,461,593; U.S. Pat. No. 5,908,613). CoQ-10 generally is solubilized in combinations with oils and surfactants, or in the essential oils used to flavor the composition. The solubilizing agents used have several disadvantages in complex formulations used in oral care products. For example, the oils impart unfavorable mouth-feel and the flavoring agents may have limited solubility of CoQ-10. The solubilizing effects of surfactants are overcome on dilution resulting in precipitation of CoQ-10.

In application Ser. No. 10/748,096 filed Dec. 30, 2003, the present inventors disclosed that complexation with natural cyclodextrins significantly improves the uptake of CoQ-10 on contact with human gingival cells, fibroblasts, and intestinal cells in vitro as compared to water dispersible liposomal CoQ-10 or solubilized CoQ-10. The inclusion complexes are water dispersible and stable in the presence of formulating agents, such as, for example, polysorbate 80. It now has been surprisingly found that such complexes can be easily incorporated at higher concentrations of CoQ-10 into oral care formulations without imparting undesirable characteristics.

### SUMMARY OF THE-INVENTION

The present invention discloses incorporation of a highly bioavailable CoQ-10 cyclodextrin complex in oral care formulations. The CoQ-10 complex of the invention is water dispersible, stable, and highly bioavailable to the cells of the oral cavity.

The CoQ-10 complex of the invention can be incorporated easily into various formulations at therapeutic concentrations of CoQ-10 by dispersing into the aqueous phase or in a sorbitol solution. In one embodiment, the oral care formulation is a toothpaste having CoQ-10 concentration at about 0.005% to about 2%. In another embodiment, the oral care composition is a mouthwash containing CoQ-10 at about 0.005% to about 1%. In a further embodiment, the complex can be incorporated into a chewing gum formulation at about 0.005 to about 0.2%. The complex also can be incorporated into devices, such as, for example, dental loops as a suspension in glycerin or any other suitable carrier.

### BRIEF DESCRIPTION OF THE DRAWING

FIG. 1 graphically portrays in vitro uptake of CoQ-10 from chewing gum by human gingival cells, as reported in Example 4, below.

This drawing will be described in more detail in connection with Example 4.

### DETAILED DESCRIPTION OF THE INVENTION

The present invention discloses incorporation of water dispersible, highly bioavailable form of CoQ-10 in oral care compositions and devices. The compositions are useful in the prevention and treatment of gingivitis and periodontitis. The efficacy of the complex was improved further by the addition of bioadhesive polymers, such as, for example, one or more of Povidone (generic name for polyvinylpyrrolidone or PVP; specifically the soluble homopolymer of N-vinyl-2-pyrrolidone), carboxymethylcellulose, or hydroxypropylcellulose.

The CoQ-10 cyclodextrin complexes containing about 20% CoQ-10 were prepared by the aqueous dispersion method followed by freeze drying (see, Ser. No. 10/748,096, cited above). The cyclodextrins include commercially available, GRAS, α-, β-, or γ-cyclodextrin. The CoQ-10 used was 98%-99% pure, as manufactured by Kaneka Company, Osaka, Japan. The CoQ-10 cyclodextrin complex was a light yellow powder, dispersible in water and stable in the presence of surfactants. The oral care formulations were representative of commercial products.

The toothpaste compositions generally contain surfactants, polishing agents, humectants, flavors, colorants, and gelling agents. They also may contain antimicrobials and anti-plaque agents. Crystalline CoQ-10 typically is dissolved in the flavor component before incorporation into the formulation. Since the amount of flavor forms a small percentage of the formulation, the CoQ-10 may precipitate out, resulting in poor uptake. It surprisingly was found that the CoQ-10 cyclodextrin complex can be incorporated into the aqueous phase as a dispersion or mixed with the surfactants before adding to the rest of the batch. The ease of incorporation makes it possible to have uniform distribution of CoQ-10 in the product and increase the concentration of CoQ-10. The concentration of CoQ-10 ranges from about 0.005% to about 2%.

The CoQ-10 cyclodextrin complex was incorporated into a mouth rinse. The composition typically contains water, surfactants, humectant, preservatives, whiteners, sweetening, and flavoring agents. The CoQ-10 complex was added along with the surfactants or to the final formulation. Surprisingly the complex formed a clear solution at the concentrations tested from 0.005% to 1%. The solution remained stable on storage without any precipitation of CoQ-10.

The complex of the present invention also was incorporated into chewing gum. The composition contained a natural or synthetic gum base, sorbitol, whitening agent, preservatives, flavor, color, sweeteners, and in some instances an antibacterial agent. The CoQ-10 complex was dispersed in sorbitol and sprayed on the gum base as a layer. The concentration of CoQ-10 was about 0.005% to about 0.2%. The chewing gum containing the complex of the invention could be dried without any moisture retention. In contrast, the same formulation containing a CoQ-10 liposome preparation instead of the cyclodextrin complex retained significant moisture even after prolonged drying. The complex also can be incorporated into lozenges and breath mints, for example as a dispersion in sorbitol. For lozenges and breath mints, the amount of CoQ-10 can range from by weight between about 0.05% and about 2% CoQ-10.

The complex of the present invention also can be incorporated into dental loops as a suspension in a suitable carrier such as, for example, glycerine or sorbitol. Dental loops can contain by weight between about 0.005% and about 0.2% CoQ-10.

While the invention has been described with reference to a preferred embodiment, those skilled in the art will understand that various changes may be made and equivalents may be substituted for elements thereof without departing from the scope of the invention. In addition, many modifications may be made to adapt a particular situation or material to the-teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. In this application all units are in the metric system and all amounts and percentages are by weight, unless otherwise expressly indicated. Also, all citations referred herein are expressly incorporated herein by reference. The following examples show how the present invention has been practiced, but should not be construed as limiting.

### EXAMPLES

### Example 1

### ToothPaste Gel Formulation

A representative toothpaste gel formulation contained water (24.13%), sorbitol (37.58%), glycerin (5%), silica (21.5%), tetrasodium pyrophosphate (2.85%), tetrapotassium pyrophosphate (2.75%), PEG-300 (2%), sodium lauryl sulfate (1.4%), Flavor (1.05%), cellulose gum (0.9%), sodium saccharin (0.25%), sodium fluoride (0.24%), sodium ascorbyl phosphate (0.1%), color (0.0036%), and CoQ-10-cyclodextrin complex (0.25%, equivalent to 0.05% CoQ-10). The CoQ-10 complex was dispersed in the water, sorbitol mixture before adding the other components.

### Example 2

### Mouthwash Formulation

A representative mouthwash formulation contained water (86.98%), glycerin (7.5%), sodium tripolyphosphate (3%), sodium benzoate (0.5%), sodium saccharin (0.06%), PEG 8000 (1%), cremophor RH-60 (0.6%), flavor (0.15%), sodium ascorbyl phosphate (0.2%), CoQ-10 cyclodextrin complex (0.025%, equivalent to 0.005%). The CoQ-10 complex was dispersed with the surfactants at 70° C., and mixed with the rest of the formulation. The mixture was maintained at 70° C. with stirring till a clear solution was obtained.

### Example 3

### Chewing Gum Formulation

A representative chewing gum formulation contained sorbitol (53%), gum base (24.94%), maltitol syrup (7.23%), xylitol (4.05%), Urea-(1.95%), peppermint oil-(0.98%), color (0.52%), sodium bicarbonate (0.51%), stevia extract (0.51%), flavor (0.34%), dicalcium phosphate (0.29%), color flakes (0.27%), sodium ascorbate (0.09%), resinous glaze (0.09%), acesulfame-K (0.06%), carnauba wax (0.04%), CoQ-10 complex (0.45% equivalent to 0.09% CoQ-10). The CoQ-10 complex was dispersed in sorbitol and sprayed onto the gum base.

### Example 4

### Uptake of CoQ-10 from the Chewing Gum In Vitro by Human Gingival Cells

The objective of the study was to demonstrate that the CoQ-10 complex remains stable during formulation and retains its properties in terms of water dispersibility and uptake at a cellular level. Normal human gingival cells were used for the uptake studies. HGF-1 cells (ATCC, Rockville, Md.) were maintained in DMEM with 10% fetal bovine serum, glutamine, and pyruvate in a humidified atmosphere at 10% CO₂ and 37° C. The cells were allowed to reach confluency in 25 cm² flasks before the start of the experiment.

The stock solution was prepared by sonicating the chewing gum (one piece, 3 g, 15 mg CoQ-10) in 10 ml of phosphate buffered saline (PBS) until the coating dispersed in solution. An aliquot of the solution was diluted in the culture medium for the experiment.

The monolayers were washed with PBS before adding the test samples at known CoQ-10 concentration (100 μg/flask). The control flasks had an equivalent amount of PBS in the medium. The cultures were incubated as before and the CoQ-10 uptake was terminated at 30 and 60 min.

The medium was removed and the monolayers were washed with PBS, followed by three washes with 5 mM sodium taurocholate in PBS. The bile salt wash removes the CoQ-10 adhering to cell surfaces. The cells were scraped into cold PBS and pelleted using centrifugation. The pellets were immediately suspended in 3 ml hexane:ethanol (50:20) and stored frozen at −20° C. until analysis. The results are the average of duplicate experiments. The pelleted cells were extracted twice with hexane:ethanol (50:20) after a freeze-thaw cycle, followed by two extractions with diethyl ether. The extracts were combined and evaporated to dryness with N₂ gas. The residue was dissolved in 0.2 ml of acetonitrile:tetrahydrofuran (55:40), and CoQ-10 content was estimated by reverse phase HPLC. Standard CoQ-10 (Sigma) was used for calibration. A Hitachi HPLC system equipped with a diode array detector and Waters Nova-Pak C-18 column, 4μ, 150×3.9 mm was used for the studies. The mobile phase was acetonitrile:tetrahydrofuran (55:40):water (93:7) at a flow rate of 1 ml/min. The chromatograms were monitored at 275 nm.

Observations

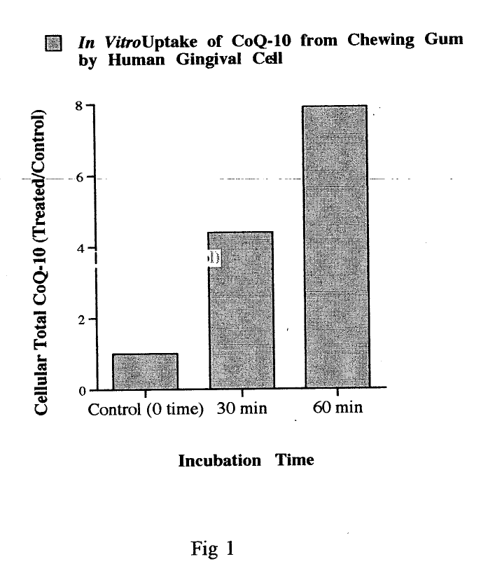

The results recorded are presented below and displayed in FIG. 1. The results indicate that the CoQ-10 complex remains stable and retains its properties after formulation. There was a fourfold to eightfold increase in the cellular CoQ-10 level after 30-60 minutes incubation, as compared to the untreated control.

TABLE 1
Uptake of CoQ-10 from Chewing Gum Extract
by Human Gingival Fibroblasts In Vitro

| Incubation Time | Cellular Total CoQ-10 |
|---|---|
| (min) | (Ratio: Treated/Control) |
| 30 | 4.40 |
| 60 | 7.90 |

We claim:

1. In an oral care formulation containing Coenzyme Q-10 (CoQ-10), the improvement which comprises:
a therapeutic concentration of CQ-10 introduced into said oral care formulation by dispersing said CoQ-10 in one or more of the aqueous phase or in the surfactant phase.

2. The improved oral care formulation of claim 1, which is one or more of:
(a) a toothpaste containing by weight between about 0.005% and about 2% COQ-10;
(b) a mouthwash containing by weight between about 0.005% and about 1% CoQ-10;
(c) a chewing gum containing by weight between about 0.005% and about 0.2% CoQ-10;
(d) a dental loop containing by weight between about 0.005% and about 0.2% CoQ-10; and
(e) lozenges and breath mints containing by weight between about 0.05% and about 2% CoQ-10.

3. The improved oral care formulation of claim 1, which further comprises a bioadhesive polymer.

4. The improved oral care formulation of claim 3, wherein said bioadhesive polymer is one or more of polyvinylpyrrolidone, carboxymethylcellulose, or hydroxypropylcellulose.

5. The improved oral care formulation of claim 4, wherein said bioadhesive polymer comprises N-vinyl-2-pyrrolidone.

6. The improved oral care formulation of claim 2, wherein said toothpaste comprises one or more of surfactants, polishing agents, humectants, flavors, colorants, a bioadhesive polymer, or gelling agents.

7. The improved oral care formulation of claim 2, wherein said mouthwash comprises one or more of water, surfactants, humectanst, preservatives, whiteners, sweeteners, a bioadhesive polymer, or flavoring agents.

8. The improved oral care formulation of claim 2, wherein said chewing gum comprises one or more of a natural gum base, a synthetic gum base, sorbitol, whitening agent, preservatives, flavor, color, sweeteners, a bioadhesive polymer, or an antibacterial agent.

9. A method for making an oral care formulation containing oral care ingredients and Coenzyme Q-10 (CoQ-10), which comprises the steps of:
(a) dispersing a therapeutic concentration of CoQ-10 in one or more of the aqueous phase or in the surfactant-phase, and
(b) adding said dispersed C-Q-10 to said oral care ingredients to make said oral care formulation.

10. The method of claim 9, wherein said oral care formulation is one or more of:
(a) a toothpaste containing by weight between about 0.005% and about 2% CoQ-10;
(b) a mouthwash containing by weight between about 0.005% and about 1% CoQ-10;
(c) a chewing gum containing by weight between about 0.005% and about 0.2% CoQ-10; and
(d) a dental loop containing by weight between about 0.005% and about 0.2% CoQ-10.

11. The method of claim 9, wherein said oral care formulation ingredients further comprises a bioadhesive polymer.

12. The method of claim 11, wherein said bioadhesive polymer is one or more of polyvinylpyrrolidone, carboxymethylcellulose, or hydroxypropylcellulose.

13. The method of claim 12, wherein said bioadhesive polymer comprises N-vinyl-2-pyrrolidone.
